(19) United States
(12) Patent Application Publication
(10) Pub. No.: US 2022/0284998 A1
(43) Pub. Date: Sep. 8, 2022

(54) INTEROPERATIVE NORMALIZED LEARNING HEALTH SYSTEM

(71) Applicant: CentiBlick Inc., Edmond, OK (US)

(72) Inventor: Kenneth Edward BLICK, Edmond, OK (US)

(73) Assignee: CentiBlick Inc., Edmond, OK (US)

(21) Appl. No.: 17/685,699

(22) Filed: Mar. 3, 2022

Related U.S. Application Data
(60) Provisional application No. 63/155,899, filed on Mar. 3, 2021.
(60) Provisional application No. 63/210,661, filed on Jun. 15, 2021.

(51) Int. Cl. G16H 10/60; G16H 15/00; G16H 40/67; G06F 3/04817

(52) U.S. Cl. CPC G16H 10/60; G16H 15/00; G16H 40/67; G06F 3/04817

(57) ABSTRACT

A method, system, apparatus, and computer program product for interoperative normalized learning health system (NLHS) disease test and scale for health and disease risk, disease monitoring, and disease diagnosis based on combined multiple normalized and weighted, organ and selected disease specific laboratory tests, vital signs/measurements, history, genetics, and observations.

### BACKGROUND

The NLHS organ and disease specific tests to include the following: 1) multiple discrete and specific quantitative and qualitative laboratory measurements on blood, other bodily fluids, tissue, cells, feces, or otherwise which may vary by sex, age, and race; 2) vital system signs and measurements which can also vary by sex, age, and race; and 3) other measurement, calculations-based body size, height, weight, diet, smoking, alcohol intake, and other like factors. 4) genetic factors and family history of disease; and 5) other measurements and observations.

### SUMMARY

A method, system, apparatus, and computer program product for interoperative normalized learning health system (NLHS) disease test and scale for health and disease risk, disease monitoring, and disease diagnosis based on combined multiple normalized and weighted, organ and selected disease specific laboratory tests, vital signs/measurements, history, genetics, and observations.

In various exemplary embodiments, a method may comprise providing a user device configured to present a graphical user interface (GUI) on a display to a user, the user comprising a physician, a medical practitioner, or a patient; providing electronic medical records (EMR) data; allowing the user device to request the EMR data; normalizing the EMR data by a processor; comparing the EMR data with one or more threshold values by the processor, wherein the comparison determines whether medical test results are within a normal range, close to boundaries of the normal range, or outside of the normal range; displaying a subset of the displayed medical information, wherein the subset is displayed differently to indicate the medical test results that are within the normal range, close to the boundaries of the normal range, or outside of the normal range.

In various exemplary embodiments, the method may further comprise transmitting, from the EMR server to the device, in response to at least one of a request provided to the EMR server by the device or an alert, a data set comprising medical information provided in the memory of the EMR server; formatting the medical data into a homogeneous format based on a predetermined preference value defining a default display; converting the medical data into one or more graph elements compatible with the GUI of the user device; displaying the normalized medical information through the GUI on the user device; prompting at least one of the physician, practitioner, or patient to take action based on the compared data via a prompt provided on the GUI; providing, in association with the prompt, a confirmation dialog, wherein further access to the GUI is secured behind the confirmation dialog; and combining the data with a user input, the user input comprising an identity of the at least one of the physician, practitioner, or patient.

In various exemplary embodiments, the medical data may comprise patient test results from a plurality of medical tests. In various exemplary embodiments, the method may further comprise generating and transmitting an alert based on the identification of a medical trend. The displayed GUI may include at least one icon associated with a subset of the transmitted medical information and which indicates at least one of a presence of a comment, a presence of additional information, or an alert. The GUI may display at least one medical test result and wherein additional medical information related to the at least one medical test result is displayed in response to user interaction with the at least one medical test result displayed on the GUI. The method may further comprise transmitting the EMR data from at least one of a cloud service or an HL7 server.

In various exemplary embodiments, a system may comprise: an electronic medical records (EMR) server having a processor and a memory, wherein the processor of the EMR server is configured to allow a user device to request data in the memory from the EMR server, normalize the data, compare the data with one or more threshold values, wherein the comparison determines whether medical test results are within a normal range, close to the boundaries of the normal range, or outside of the normal range, transmitting, from the EMR server to the user device, in response to at least one of a request provided to the EMR server by the user device or an alert, a data set comprising medical information provided in the memory of the EMR server, format the medical data into a homogeneous format based on a predetermined preference value defining a default display, and convert the medical data into one or more graph elements; and the user device is configured to present a graphical user interface (GUI) on a display to a user, display the normalized medical information through the GUI, prompt taking action based on the compared data via a prompt provided on the GUI, provide, in association with the prompt, a confirmation dialog, wherein further access to the GUI is secured behind the confirmation dialog, and combine the data with a user input, the user input comprising an identity of the user.

In various exemplary embodiments, the medical data may comprise patient test results from a plurality of medical tests. The processor of the EMR server may be further configured to display a subset of the displayed medical information, and the subset is displayed differently to indicate the medical test results that are within the normal range, close to the boundaries of the normal range, or outside of the normal range. The processor of the EMR server may be further configured to generate and transmit an alert based on the identification of a medical trend. The user device may be further configured to display at least one icon, associated with a subset of the transmitted medical information, indicating at least one of a presence of a comment, a presence of additional information, or an alert. The user device may be further configured to displays at least one medical test result and display additional medical information related to the at least one medical test result in response to user interaction with the at least one medical test result displayed on the GUI. The server may be one of an HL7 fire server or a cloud service.

### BRIEF DESCRIPTION OF THE DRAWINGS

Advantages of exemplary embodiments will be apparent from the following detailed description. The following detailed description should be considered in conjunction with the accompanying figures in which:

FIG. 1 is an exemplary embodiment depicting displayed content on four screens;

FIG. 2 is an exemplary embodiment related to FIG. 1;

FIG. 3 is an exemplary embodiment related to FIG. 1;

FIG. 4 is an alternative embodiment related to FIG. 1; and

FIG. 5 is a GUI of an exemplary embodiment of software described herein.

### DETAILED DESCRIPTION

Aspects of exemplary embodiments are disclosed in the following description and related drawings. Those skilled in the art will recognize that alternate embodiments may be devised without departing from the spirit or the scope of the claims. Additionally, well-known elements of exemplary embodiments will not be described in detail or will be omitted so as not to obscure the relevant details. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the term “embodiments” does not require that all embodiments include the discussed feature, advantage, or mode of operation.

Further, many of the embodiments described herein may be described in terms of sequences of actions to be performed by, for example, elements of a computing device. It should be recognized by those skilled in the art that the various sequence of actions described herein can be performed by specific circuits (e.g., application specific integrated circuits (ASICs)) and/or by program instructions executed by at least one processor. Additionally, the sequence of actions described herein can be embodied entirely within any form of computer-readable storage medium such that execution of the sequence of actions enables the processor to perform the functionality described herein. Thus, the various embodiments may be embodied in a number of different forms, all of which have been contemplated to be within the scope of the claimed subject matter. In addition, for each of the embodiments described herein, the corresponding form of any such embodiments may be described herein as, for example, “a computer configured to” perform the described action.

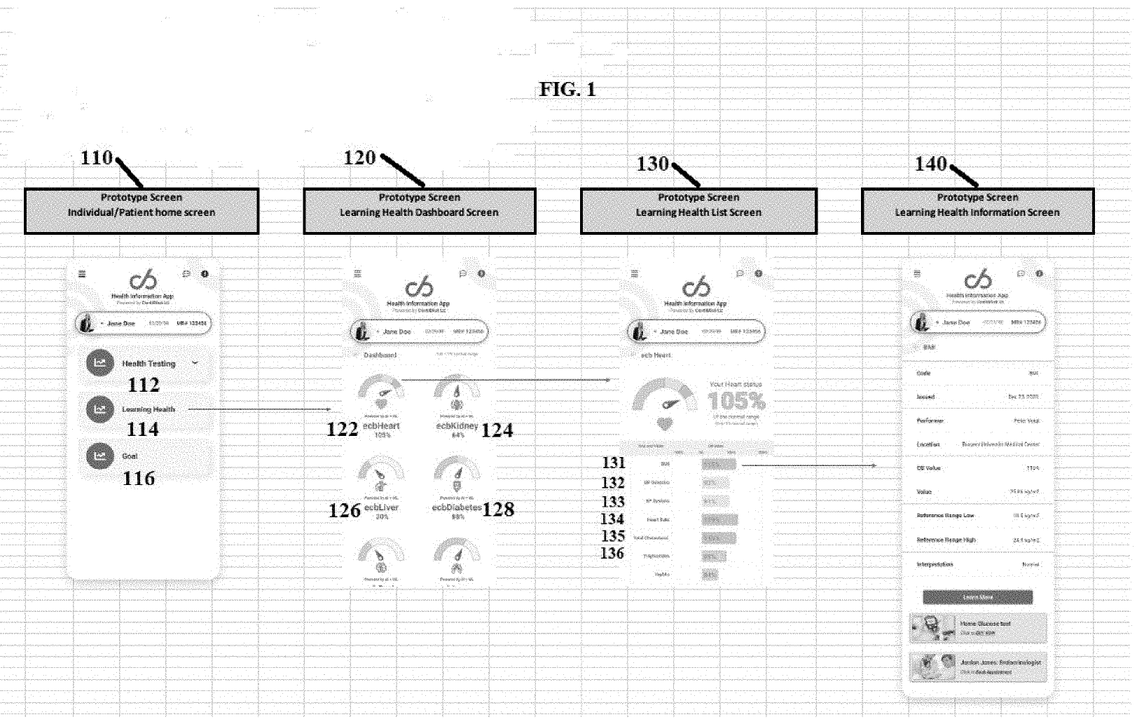

As shown in FIG. 1, an exemplary embodiment may display content on four prototype screens. The first prototype screen 110 may be the patient's home screen. The second prototype screen 120 may be a learning health dashboard screen. The third prototype screen 130 may be a learning health list screen. The fourth prototype screen 140 may be a learning health information screen.

The first prototype screen 110 may be described as Health Information App for a particular user. It may be a home screen for an individual patient. In the exemplary embodiment, a particular user, Jane Doe, may be identified by name, birth date, and by an identification number. Three options may appear below the identifying information: Health Testing 112, Learning Health 114, and Goal 116. Selecting Learning Health 114 activates the second prototype screen 120.

The second prototype screen 120 repeats the identifying information from the first prototype screen 110. In this exemplary embodiment, four displays are visible: ecbHeart 122, ecbKidney 124, ecbLiver 126, and ecbDiabetes 128. The value for ecbHeart 122 is 105% of the normal range, so an alert signal may be displayed in a red color. Selecting this alert signal will activate the third prototype screen 130. The value for ecbKidney 124 is 64% of the normal range, so no alert or caution signals are present. The value for ecbLiver 126 is 30% of the normal range, so no alert or caution signals are present. The value for ecbDiabetes 128 is 88% of the normal range, so a caution signal may be displayed in a yellow color

The third prototype screen 130 repeats the identifying information from the first prototype screen 110. As described above, the third prototype screen 130 may be triggered by selecting an item, such as ecbHeart 122, with an alert signal in the second prototype screen 120. The third prototype screen 130 displays further information related to this alert signal.

In an exemplary embodiment, the third prototype screen 130 may display BMI 131, diastolic blood pressure (BP) 132, systolic BP 133, heart rate 134, total cholesterol 135, triglycerides 136, and other parameters. BMI 131 is 115% of normal, heart rate 134 is 119% of normal, and total cholesterol 135 is 115% of normal. Thus, BMI 131, heart 134, and total cholesterol 135 all have alert signals displayed in red. Clicking on the alert signal for BMI 131 may trigger the fourth prototype screen 140. Diastolic BP 132 is 92% of normal and systolic BP 133 is 91% of normal. Thus, diastolic BP 132 and systolic BP 133 have caution signals displayed in yellow. The triglycerides 136 have neither alert nor caution signals.

The fourth prototype screen 140 repeats the identifying information from the first prototype screen 110. As described above, the fourth prototype screen 140 may be triggered by selecting an item, such as BMI 131, with an alert signal in the third prototype screen 130. The fourth prototype screen may provide additional information about BMI 131.

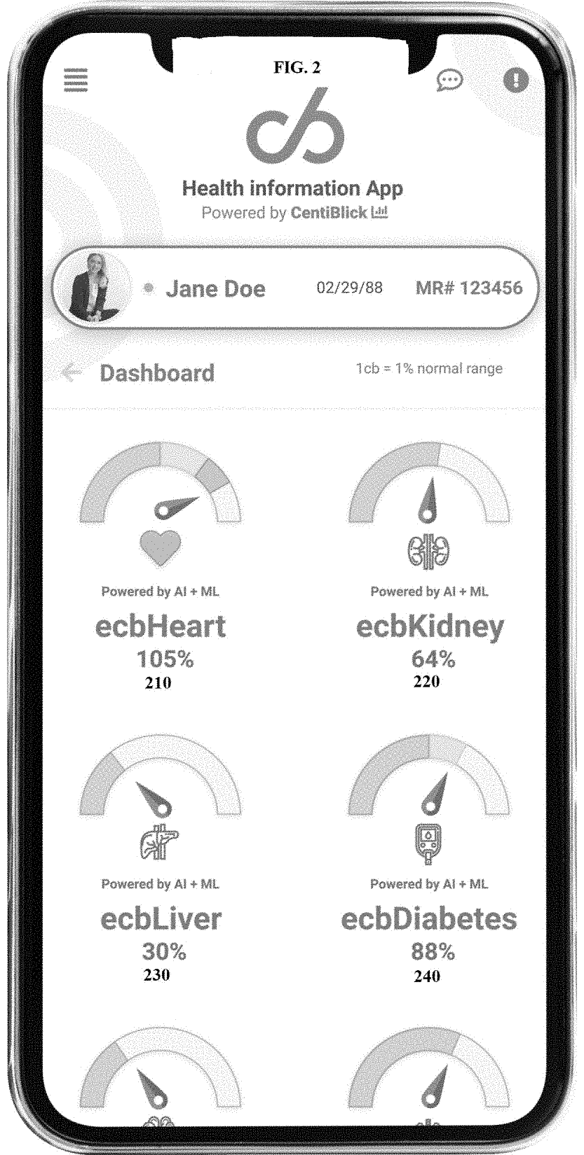

FIG. 2 depicts an exemplary embodiment that may correspond to the second prototype screen 120 of FIG. 1. In this exemplary embodiment, four displays are visible: ecbHeart 210, ecbKidney 220, ecbLiver 230, and ecbDiabetes 240. The value for ecbHeart 210 is 105% of the normal range, so an alert signal may be displayed in a red color. Selecting this alert signal will activate the third prototype screen 130. The value for ecbKidney 220 is 64% of the normal range, so no alert or caution signals are present. The value for ecbLiver 230 is 30% of the normal range, so no alert or caution signals are present. The value for ecbDiabetes 240 is 88% of the normal range, so a caution signal may be displayed in a yellow color

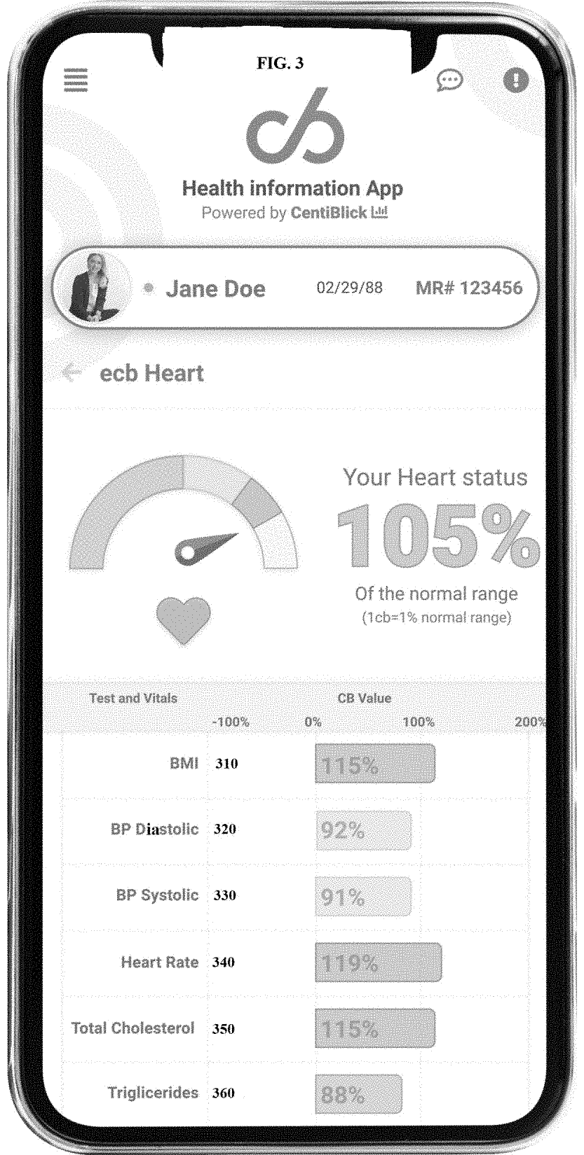

FIG. 3 depicts an exemplary embodiment that may correspond to the third prototype screen 130 of FIG. 1. FIG. 3 may display BMI 310, diastolic BP 320, systolic BP 330, heart rate 340, total cholesterol 350, and triglycerides 360.

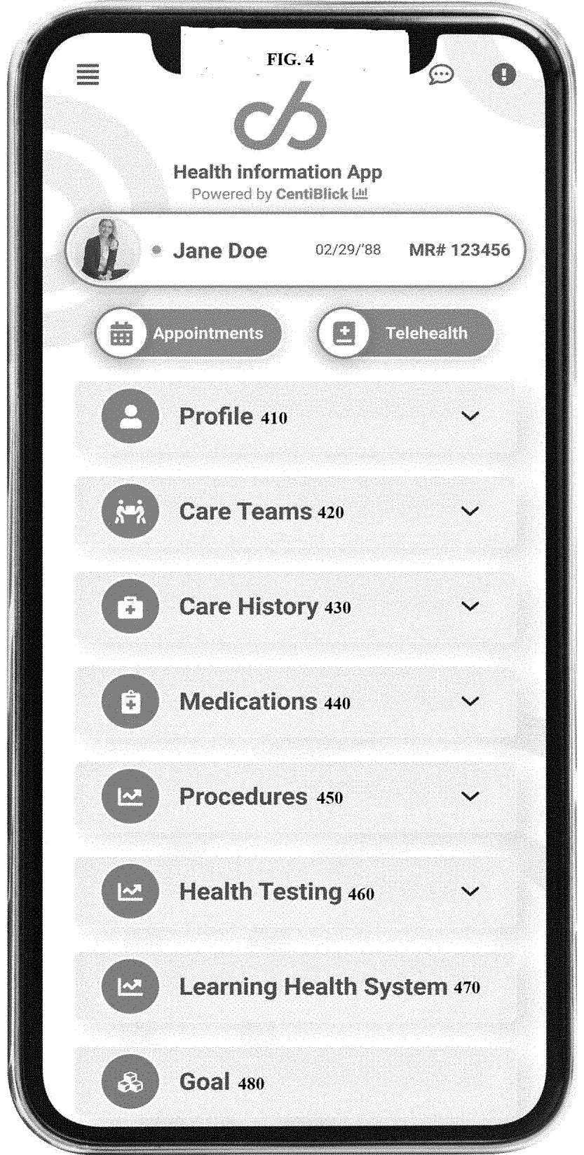

FIG. 4 may correspond to an alternative embodiment of the first prototype screen 110 of FIG. 1. It may be a home screen for an individual patient. In the exemplary embodiment, a particular user, Jane Doe, may be identified by name, birth date, and by an identification number. Many options may appear below the identifying information: Profile 410, Care Teams 420, Care History 430, Medications 440, Procedures 450, Health Testing 460, Learning Health System 470, and Goal 480. In addition, FIG. 4 may provide options for making appointments 482 and for using telehealth 484.

In the exemplary embodiments, selection of multiple healthcare measurements which have been showed to have positive or negative correlation with the presence or absence of disease or disease risk to be combined into a single NLHS Disease test parameter.

The normalized CentiBlick scale reporting for the NLHS test is as follows:

−100 to 0, Low abnormal test result;

0 to 10, Borderline Low test result;

20 to 80 Normal test result;

80 to 100 Borderline High test result; and

>100 High abnormal test result. Also see U.S. Pat. No. 8,204,713, the contents of which are incorporated by reference in their entirety.

NLHS test results for disease may be reported numerically, graphically, alphabetically, or using other symbols or nomenclature. In various embodiments, horizontal or vertical color bars may be used, with green bars for normal test results, yellow bars for borderline test results, and red bars for high test results. Other colors may also be used as determined.

For multiple NLHS results done at different times, horizontal bars may be stacked, in an exemplary embodiment, chronologically in bar clusters with the most recent NLHS test result being on the top and the first on the bottom bar stack or cluster but may also be stacked, filtered, or displayed in various other orders and manners.

Multiple numeric tests, vitals, and other measurements/observations with sex, age, and race established reference ranges are normalized to one scale, as disclosed in U.S. Pat. No. 8,204,713, and combined numerically into one NLHS test parameter based on at least one of all of the following.

a. A weighted multiplier based on the published predictive value of each test or measurement and other tests included in the organ/disease specific NLHS test;

b. A Learning Health System modifier weighting factor which will be adjusted statistically based on new studies periodically refining the goodness of fit of NLHS with actual observations and practice.

c. The Learning Health System weighting factor which will be adjusted based on artificial intelligence (AI), machine learning (ML), or an algorithm refining the goodness or properness of fit of NLHS.

As the ecbTest may use a normalized value, it may approximate the Gaussian and non-Gaussian distribution test results enough to make any difference clinically insignificant. Moreover, the actual reported test results with reporting units may be available with a touch or click drilldown if the user wants access to the actual reported test results.

A lab or vital test has a documented “Predictive Value” for disease. For example, a 100 percent predictive value may mean that a positive test is 100 percent diagnostic for a disease. This may also be expressed as a probability of 1.0. Such probability factors may be used as the weighted multipliers.

Conversely, a negative test may indicate a low probability of disease and have a negative predictive value. In this case, the weighted multiplier may approach zero. The probability would correspond to low risk for a disease on an ecbTest.

Each test parameter to be included in the NLHS organ/disease specific test is calculated as follows:

Normalized Learning Health System test weight factor (lhsWF) initially set to 1.0 for each test to be included:

a. Predictive Value Weight Factor (pvWF) for each included health test parameter

b. Normalized health measurement or observation based on the patented CentiBlick system (cbTest)

c. Each cbTest is then added with other tests and divided by the number of tests to compute a new normalized NLHS ecbDisease test as follows (ecbRenal given as an example below):

ecbRenal=((lhsWF)×(pvWF)×(cbTest1)+(lhsWF)×(pvWF)×(cbTest2)+(lhsWF)×(pvWF)×(cbTest3))/3 i.

with addition of other normalized tests as appropriate

Initially all lhsWf=1.00 (to be adjusted based on health statistical correlation disease studies in selected populations and groups.)

pvWF is computed as based on 1) published test Predictive Value for Receiver Operator Curves (ROC) curves for determination Area Under Curve (AUC) and 2) divided by the average of all AUC values included in the ecbDisease test. This pvWF parameter will be set to 1.00 if no suitable and reliable predictive value statistics are available in the literature.

AUC computations may involve published values from research on a test by plotting the True Positives for disease Value Rate (test sensitivity for a disease as a decimal) versus the False Positive Test Rate (1-Specificity) for the disease. Both true positives and false positives may be scaled over a number of test values. These two variables may be plotted graphically. Then the area of the resulting curve may be integrated to get the area under curve (AUC).

When AUC is not available or less desirable statistically, the pvWF for each test or measurement value included can be computed by 1) adding the test diagnostic sensitivity and specificity as probabilities (decimals) and dividing by 2.0 and 2) dividing all resulting values by the average of all tests included in the ecbDisease test. If acceptable disease sensitivity and specificity values are not available in the literature, the pvWF will be set to 1.00.

pvWF for each test or measurement can also be computed or adjusted by Relative Risk of disease computed 1) by dividing the sensitivity as a probability/decimal by one minus the specificity and then 2) dividing by the average Relative Risk of all test parameters included in the ecbTest.

The normalized CentiBlick value for each test is computed and scaled using the existing US patented equations and scaling.

The ecbTests include ecbRenal, ecbHeart, ecbLiver, ecbLung, ecbDiabetes, ecbCancer, ecbCoagulation, ecbInflamation, ecbEnergy, ecbEndocrine, ecbGastrointestinal, ecbCNS.

a. Normalized cbTests to compute NHLS ecbRenal include the following:

Creatinine, Blood Urea Nitrogen, Creatinine Clearance, Fasting Glucose, Glycosuria, Hemoglobin A1c, Urine Microalbumin, ecbDiabetes, Proteinuria Screen, and other tests as indicated.

b. Normalized cbTests to compute NHLS ecbHeart include the following:

Total Cholesterol, Triglyceride, Basic Metabolic Rate (BMI), Blood Pressure (systolic and diastolic), Heart Rate, Respiratory Rate, LDL-Cholesterol, HDL-Cholesterol, LDL/HDL Cholesterol Ratio, Ultra Sensitive Troponin, Hemoglobin A1c, hsCRP, ecbDiabetes, cbAge, alcohol, and other tests as indicated.

c. Normalized cbTests to compute NHLS ecbLiver include the following:

Aspartate Aminotransferase (AST), Alanine Aminotransferase (ALT), Bilirubin (Direct and Indirect), Blood Ammonia, Albumin Globulin Ratio, Alkaline Phosphatase, Gamma-glutamyl Transferase, Alpha 1 Antitrypsin, BMI, Triglyceride, ecbDiabetes, Hemoglobin A1c, and other tests as indicated.

d. Normalized cbTests to compute NHLS ecbLung include the following:

Blood Oxygen Saturation, Blood Partial Pressure of Oxygen and Carbon Dioxide, Forced Expiratory Volume 1, cbSmoker, Alpha 1 Antitrypsin, Body Mass Index, Peak Expiratory Flow Rate. cbRespiratory Rate, cbHeart Rate, and other tests as indicated.

e. Normalized cbTests to compute NHLS ecbDiabetes include the following:

Fasting Glucose, Hemoglobin A1c, Triglyceride, BMI, AST. Microalbumin, Glucosuria, Proteinuria, and other tests as indicated.

f. Normalized cbTests to compute NHLS ecbCancer include the following:

cbSmoker, Occult Stool Blood, CA-125, CA15-3, Ca19-9, CEA, PSA (males), HPV antibody, BMI, Sex, cbAge, cbHistory, and other tests as indicated.

g. Normalized cbTests to compute NHLS ecbCoagulation include the following: Prothrombin Time, Partial Thromboplastin Time, Platelet Count, D-Dimer. cbAnticoagulant, cbFactor Analysis. cbBleeds/Clots History, and other tests as indicated.

h. Normalized cbTests to compute NHLS ecbInflammation include the following: CRP, Sedimentation Rate, White Blood and Lymphocyte Cell Count, cbTemperature, cbHeart Rate, and other tests as indicated.

i. Normalized cbTests to compute NHLS ecbEnergy include the following: Hemoglobin, Hematocrit, FreeT4, Thyroid Stimulating Hormone, Serum Iron, Transferrin, Cortisol, Testosterone, White Cell Count, CRP, and other tests as indicated.

j. Normalized cbTests to compute NHLS ecbEndocrine include the following:

FreeT4. Thyroid Stimulating Hormone, Cortisol, Free Testosterone, Estradiol, Beta HCG, and other tests as indicated.

k. Normalized cbTests to compute NHLS ecbGastrointestinal include the following: cbFecal Occult Blood, Helicobacter Pylori Antibody, Stool H. Pylori Antigen, Gastrin, cbNSAID, cbChewingGum, cbEndoscopy, cbDiet, and other tests as indicated.

l. Normalized cbTests to compute NHLS ecbBrainCNS include the following:

Porphyromonas gingivalis. hsCRP, Procalcitonin, Campylobacter rectus, and Prevotella melaninogenica, Brain CT Scan, cbCognitive Test, and other tests as indicated.

All NHLS ecbDisease and ecbOrgan tests can be combined into one interoperable, normalized test parameter by adding each specific ecbDisease and ecbOrgan tests and dividing by the total number of tests included to get the average (See below for an example computation):

a. ecbDisease Relative Risk=(ecbRenal+ecbHeart+ecbLiver+ecbLung+ecbDiabetes+ecbCancer+ecbCoagulation+ecbInflammation+ecbBrainCNS)/9

Elements of U.S. Pat. No. 8,204,713 describe some exemplary aspects of this description in more detail. Additionally, exemplary embodiments described herein may include physician, practitioner, and/or patient interactive software for the viewing, conveying, sharing, and communicating of medical results. The software can include a dashboard and/or user interface for reporting any desired medical information on a computer display station, personal computer, and mobile wireless devices including handheld smartphones, tablets, and the like, which may generally be considered as user devices. It may further be appreciated that such software can be utilized on any device with display and network connectivity capabilities. Exemplary embodiments of software described herein may further have graphical displays, for example color graphical displays, and normalization of numerical information as described in the incorporated by reference U.S. Pat. No. 8,204,713. As described in detail in U.S. Pat. No. 8,204,713, medical information may be normalized by converting raw data to a decimal equivalent scale that facilitates reporting and analyzing the data.

Further, the software can facilitate physician, practitioner, and/or patient reporting and review of any desired types of medical information found in a typical medical chart (or electronic medical records system) with all results on the same normalized scale, in any desired numerical and non-numerical format. In an exemplary embodiment, the scale may be a CentiBlick Result (CBR) normalized graphic scale. A normalized graphical interface between a user and physician can provide data access, viewing and communication capabilities via click or touch screen functionality, as well as through any other user-device interaction, for example via stylus, mouse, voice command, or similar methods. The exemplary software may further present any desired medical information in a standard horizontal bar graph, as shown in more detail below with respect to the figures. Such views can include vertical and horizontal scrolling and various identifying features such as shading, patterns, or color graphics; for example, abnormal or positive findings may be presented as a red bar, borderline or indeterminate findings may be presented as a yellow bar, and findings or results in the “normal range” or desired range of are presented as a green bar. Other colors or visual representations and other data sets may also be included; for example, necropsy/autopsy reports may be presented as a blue bar. It can be appreciated that the colors used here are merely exemplary and any other desired color or visual indicia may be utilized to provide or convey information. Alternative graphical displays, for example, vertical bars or another desired orientation may also be selected depending on the user's preference. This may ensure that the data scales to the available display space or can otherwise be displayed as desired. Other types of graphs or charts may also be utilized.

Still referring to the exemplary embodiments described herein, test results on the same test may be stacked into a data bar graph cluster with the most recent test being displayed on the top bar of the associated bar graph test cluster. According to at least one embodiment, a physician (or any other party) can elect in in the software preferences menu or elsewhere to have the most recent result at the bottom of the cluster, or to change the orientation or visual indicia in any desired fashion. This test cluster reporting can allow for a current test result shown on a top bar to be trended with previous results on this test, for example, by viewing the bar graph cluster showing previous results on the bars below. As one exemplary option, a physician can elect to have a line graph drawn tracing the displayed bar graph. By touching or clicking the data bar or otherwise interacting with the graphical display, the software may access and display data in the form of a traditional medical chart (for example, laboratory, vitals, and the like may be displayed in traditional form). Any of the following may be able to be displayed: standard, traditional reporting units, and laboratory method (e.g., Core Lab enzymatic, Point-of-Care (POC) i-Stat, etc. along with a corresponding normalized unit); comments regarding a reported result or results (for example a lab comment “hemolyzed sample”); a text report or medical image (for example a pathology report with a positive or negative cancer finding); a screening result (for example urinalysis); and/or a critical result requiring a physician's attention. Icons may be displayed on the device using the software that may also alert a physician that there are comments and additional details; alternatively, comments may be directly displayed without requiring a point and click or other selection. There may also be a critical result icon on the graphic display. Note that a physician, practitioner, and/or patient may be prompted to acknowledge that a critical result, medical report, or medical image was viewed using the above-described access option in order to return to the original graphical survey display. The date and time of a physician's review of the data, as well as any acknowledgements, may be recorded and displayed. A check or other indicia may be displayed next to the critical result flag, once viewed. Such a feature may be implemented using data from an electronic medical record (EMR) documenting the order entry and review loop cycle.

In still further exemplary embodiments, the system and software can also feature connectivity with medical records systems using HL7 standard formats (or any other desired format or formats) for information interchange using a push system approach for acute care critical results reporting and a user inquiry approach where a clinician/practitioner user is prompted to specify the inquiry parameters regarding a specific patient or group of patients. The push feature can send notifications to a mobile device and may allow for stacking notifications when multiple critical reports are sent. The physician, practitioner, and/or patient may then touch a notification icon (or use any other access capability) to view the complete message or report. Further, it may be appreciated that a direct connection to an HL7 fire server may be utilized. Data points coming from personal devices or third party apps, such as body weight or glucose measurements, may be uploaded directly into an app via Bluetooth short-range wireless technology or other means.

In further exemplary embodiments, test results can be listed on a scrollable color bar graph display, for example in alphabetical order by test/procedure or specimen name (e.g., uterus), or through any other criteria desired. Test/report filters may be available when selecting this option, and a physician can filter the displayed color survey bar graph into any of a variety of formats, for example organ specific test/report panels (e.g., renal, liver, cardiovascular, uterus, etc.) in order to facilitate the diagnosis and/or to assist in monitoring the course of treatment, test/report panels designed for consults in a particular specialty area, and/or test/report panels designed for facilitating the generation of a discharge summary or encounter summary. With regard to discharge and encounter summaries, the system can aggregate various panels and data sets, looking for trends and progression during the course of care based on set statistical guidelines, describe the various data events observed by the expert software, and then aggregate these findings into a narrative report, along with diagnostic codes. The latter report can include options to allow the physician, practitioner, and/or patient to review and edit this computer generated summary.

In an exemplary embodiment, a user may access a variety of options in the software using a double tap or any other selection technique. For example, a double tap of a mobile device screen displaying the software can display a menu of options including Confirm, Order Tests/Procedures, Call Floor, Call Patient, Email Patient, Text Patient, Release to Patient, Return, or other similar options. Also, with a double tap or other selection, a “Physician Preference” feature can be set to allow the default laboratory results to be viewed in any of a variety of formats, for example formatted to non-CBR, non-graphically displayed results. Using this option, laboratory results, vitals, etc. can be viewed in the traditional, legacy spreadsheet format listing traditional values, units, flags, and normal/reference ranges.

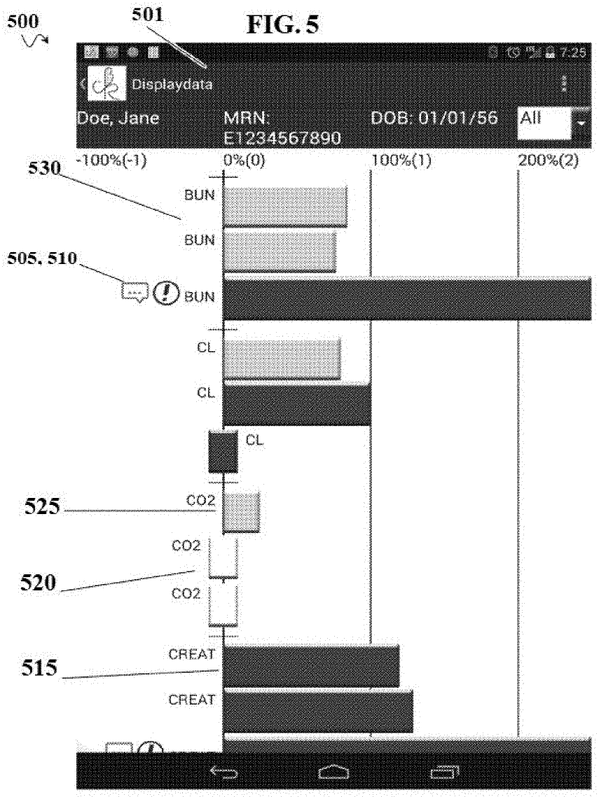

Referring now to exemplary FIG. 5, a GUI of a mobile device illustrating the display 500 of normalized laboratory results using the normalized CBR system with the software described herein may be provided. Display header 501 may include, for example, the patient's name, the patient's date of birth, and other pertinent information; other information may be displayed elsewhere on the display, as desired. Icons or other attention-grabbing items may be incorporated as part of the display, for example to identify anomalous laboratory results or to identify patients in urgent need of care. Note, in this example, that critical results can include a “!” icon 505 and comments attached to the result can be indicated with a message icon 510. The user may be able to call up more information about why these or other icons are present, for example by selecting the result bar associated with the icons 505, 510 via touch screen or by mousing over the result bar, by clicking the result bar, by touching or mousing over other result bars 515, 520, 525 with which they are associated, or otherwise communicating selection information to the device. Abnormal results may be shown with a uniquely identifying color, such as red, as shown in bar 515. Borderline results may be shown with a different color, such as yellow, as shown in bar 520. Normal results may be shown with a different color, such as green, as shown in bar 525.

Results on the same test, such as BUN, may be stacked or aggregated in the display; the exemplary embodiment of FIG. 1 displays these results in a result cluster 530, with the most recent test displayed on the topmost bar. The stacked bar cluster then shows previous normalized results below the top bar listed in chronological order thus enabling the user to follow whether a particular test/result is trending higher or lower compared to the previous bar shown below.

Trends in results may be readily observed by a user. Where applicable, trends may also be identified by the software, either automatically or following an identification procedure that a user may execute, as desired. Trends for a particular result may also be noted by artificial intelligence and machine learning and when significant, the user could be alerted that a recent result may be cause for action or concern.

A Graphical User Interface (GUI) may display at least one medical test result. Additional medical information related to the at least one medical test result may be displayed in response to user interaction with the at least one medical test result displayed on the GUI.

It should be understood that all of the embodiments and examples described herein are merely exemplary and should be considered as non-limiting.

The foregoing description and accompanying figures illustrate various exemplary embodiments but should not be construed as being limited to the particular embodiments discussed above. Additional variations of the embodiments discussed above will be appreciated by those skilled in the art. Therefore, the above-described embodiments should be regarded as illustrative rather than restrictive. Accordingly, variations to those embodiments can be made by those skilled in the art without departing from the scope as defined by the following claims.

What is claimed is:

1. A method comprising:
providing a user device configured to present a graphical user interface (GUI) on a display to a user, the user comprising a physician, a medical practitioner, or a patient;
providing electronic medical records (EMR) data;
allowing the user device to request the EMR data;
normalizing the EMR data by a processor;
comparing the EMR data with one or more threshold values by the processor, wherein the comparison determines whether medical test results are within a normal range, close to boundaries of the normal range, or outside of the normal range;
displaying a subset of the displayed medical information, wherein the subset is displayed differently to indicate the medical test results that are within the normal range, close to the boundaries of the normal range, or outside of the normal range;
transmitting, from the EMR server to the user device, in response to at least one of a request provided to the EMR server by the user device or an alert, a data set comprising medical information provided in the memory of the EMR server;
formatting the medical data into a homogeneous format based on a predetermined preference value defining a default display;
converting the medical data into one or more graph elements compatible with the GUI of the user device;
displaying the normalized medical information through the GUI on the user device;
prompting at least one of the physician, the practitioner, or the patient to take action based on the compared data via a prompt provided on the GUI;
providing, in association with the prompt, a confirmation dialog, wherein further access to the GUI is secured behind the confirmation dialog; and
combining the data with a user input, the user input comprising an identity of the at least one of the physician, the practitioner, or the patient.

2. The method of claim 1, wherein the medical data comprises patient test results from a plurality of medical tests.

3. The method of claim 1, further comprising:
generating and transmitting an alert based on the identification of a medical trend.

4. The method of claim 1, wherein the displayed GUI includes at least one icon associated with a subset of the transmitted medical information and which indicates at least one of a presence of a comment, a presence of additional information, or an alert.

5. The method of claim 1, wherein the GUI displays at least one medical test result and wherein additional medical information related to the at least one medical test result is displayed in response to user interaction with the at least one medical test result displayed on the GUI.

6. The method of claim 1, further comprising:
transmitting the EMR data from at least one of a cloud service or an HL7 server.

7. A system comprising:
an electronic medical records (EMR) server having a processor and a memory, wherein the processor of the EMR server is configured to allow a user device to request data in the memory from the EMR server, normalize the data, compare the data with one or more threshold values, wherein the comparison determines whether medical test results are within a normal range, close to boundaries of the normal range, or outside of the normal range, transmitting, from the EMR server to the user device, in response to at least one of a request provided to the EMR server by the user device or an alert, a data set comprising medical information provided in the memory of the EMR server, format the medical data into a homogeneous format based on a predetermined preference value defining a default display, and convert the medical data into one or more graph elements; and
the user device is configured to present a graphical user interface (GUI) on a display to a user, display the normalized medical information through the GUI, prompt taking action based on the compared data via a prompt provided on the GUI, provide, in association with the prompt, a confirmation dialog, wherein further access to the GUI is secured behind the confirmation dialog, and combine the data with a user input, the user input comprising an identity of the user.

8. The system of claim 7, wherein the medical data comprises patient test results from a plurality of medical tests.

9. The system of claim 8, wherein the processor of the EMR server is further configured to display a subset of the displayed medical information, and the subset is displayed differently to indicate the medical test results that are within the normal range, close to the boundaries of the normal range, or outside of the normal range.

10. The system of claim 7, wherein the processor of the EMR server is further configured to generate and transmit an alert based on the identification of a medical trend.

11. The system of claim 7, wherein the user device is further configured to display at least one icon, associated with a subset of the transmitted medical information, indicating at least one of a presence of a comment, a presence of additional information, or an alert.

12. The system of claim 7, wherein the user device is further configured to display at least one medical test result and display additional medical information related to the at least one medical test result in response to user interaction with the at least one medical test result displayed on the GUI.

13. The system of claim 7, wherein the server is one of an HL7 fire server or a cloud service.
